(19) United States
(12) Patent Application Publication
(10) Pub. No.: US 2024/0325787 A1
(43) Pub. Date: Oct. 3, 2024

(54) IMAGE PROCESSING TO SUPPORT RADIATION THERAPY WORKFLOW STEPS

(71) Applicant: Siemens Healthineers International AG, Steinhausen (CH)

(72) Inventors: Michal Walczak, Ennetbaden (CH); Pascal Paysan, Basel (CH); Mathieu Plamondon, Glattbrugg (CH); Stefan Scheib, Waedenswil (CH)

(21) Appl. No.: 18/192,915

(22) Filed: Mar. 30, 2023

(51) Int. Cl. A61N 5/10; G16H 20/40; G16H 30/40

(52) U.S. Cl. CPC A61N 5/1039; G16H 20/40; G16H 30/40; A61N 2005/1041

(57) ABSTRACT

A control circuit accesses projection data for a given patient (such as cone-beam computed tomography images). The control circuit can then background process the projection data to generate a plurality of different images. The control circuit then stores these images to provide stored images. Upon receiving a request that corresponds to at least one item of task-supportive content that corresponds to a particular radiation therapy workflow step, the control circuit may then access the aforementioned stored images to select at least one particular image that corresponds to the particular radiation therapy workflow step. That at least one particular image can then be transmitted, for example, to the functionality that requested (or that otherwise requires and is awaiting) this content.

### TECHNICAL FIELD

These teachings relate generally to treating a patient's planning target volume with energy pursuant to an energy-based treatment plan and more particularly to preparing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

A radiation treatment plan may be derived via a corresponding radiation therapy workflow comprised of a plurality of workflow steps (at least many of which are executed in a serial fashion, some steps requiring input from one or more previous steps). Some of these workflow steps may require particular kinds of images corresponding to the patient. For example, high-contrast Hounsfield Unit images may be useful or even necessary for delineation while relative electron density images may be appropriate for dose calculations. Many such images may comprise differently processed versions of patient images that were originally obtained using computed tomography and/or cone-based computed tomography. The amount of time required to prepare a given image at the time the image is needed by a particular workflow step can be noticeable and can serve to generally slow down the entire process. Present techniques also typically allow for considerable inconsistencies that are at least partially owing to different image-capture modalities that source the original patient images.

### BRIEF DESCRIPTION OF THE DRAWINGS

The above needs are at least partially met through provision of the image processing apparatus and method to support a radiation therapy workflow step described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings; and

FIG. 3 comprises a block diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments a control circuit is configured to access projection data for a given patient. These teachings will accommodate a variety of different kinds of projection data, including, either in whole or in part, cone-beam computed tomography images. The control circuit then background processes the projection data to generate a plurality of different images. The latter may comprise, for example, background processing the projection data during a radiation therapy workflow process. By one approach, this background processing comprises, at least in part, selecting a plurality of reconstruction methods from amongst a plurality of available reconstruction methods. Accordingly, as desired, some or all of the images may comprise reconstructed images while some or all of the images may comprise raw (i.e., non-reconstructed) images.

These teachings will accommodate a wide variety of image types including, but not limited to, least one, three, five, or any selected number of:
an image presenting directly reconstructed relative electron density for photon treatment dose calculation and for kV imaging dose calculation;
an image presenting directly reconstructed stopping power ratio for proton or electron treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use; and an image highlighting at least one patient implant.

The control circuit then stores different images to provide stored images. Upon receiving a request that corresponds to at least one item of task-supportive content that corresponds to a particular radiation therapy workflow step, the control circuit may then access the aforementioned stored images to select at least one particular image that corresponds to the particular radiation therapy workflow step. That at least one particular image can then be transmitted, for example, to the functionality that requested (or that otherwise requires and is awaiting) this content. By one approach, the provided image comprises a consistent representation of the task-supportive content regardless of what image-capture modality was employed to capture the projection data used to generate that image.

These teachings are both practical and flexible in application and will accommodate various modifications and/or additional steps, activities, or functionality. As one example in these regards, the control circuit can be further configured to select a particular imaging protocol for a cone-beam computed tomography imaging system from amongst a plurality of available imaging protocols, such that the projection data is captured as a function of the particular imaging protocol. By another approach, in lieu of the foregoing or in combination there with, the control circuit may be further configured to post-process the at least one particular image that corresponds to the particular radiation therapy workflow step to generate at least one post-processed particular image. In this case, the transmitted image may include or constitute that post-processed particular image.

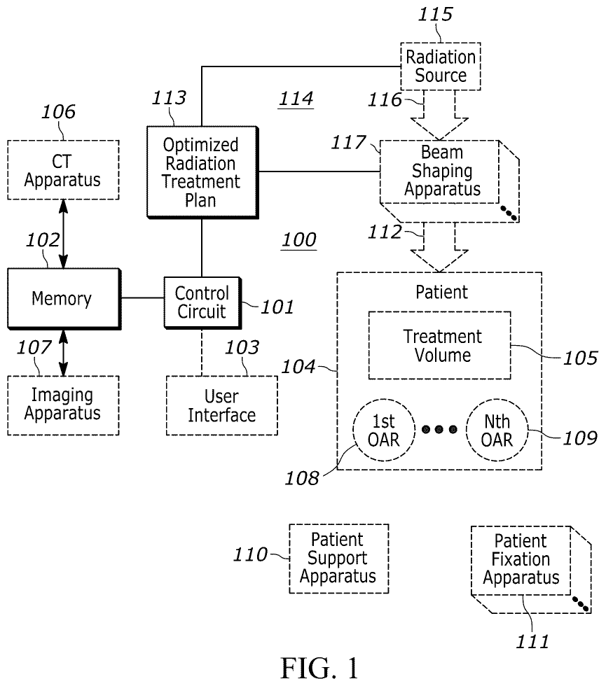

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as optimization information for a particular patient, information regarding a particular radiation treatment platform as described herein, and the projection data, stored images, and post-processed images that are described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

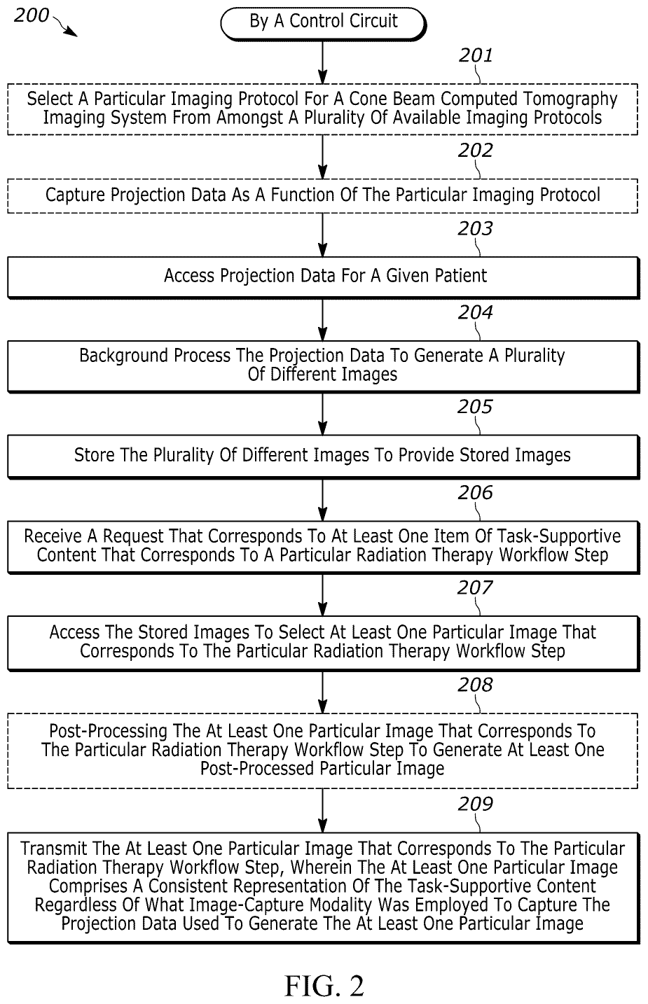

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

At optional block 201, this process 200 can select a particular imaging protocol, such as for a cone-beam computed tomography imaging system, from amongst a plurality of available imaging protocols. This selection may be based, for example, on specific or inferred user intent and/or a patient's characteristics. As one example in these regards, image acquisition parameters could be optimized so that the resulting reconstructions would be of the best quality to perform a particular task. The aforementioned protocols may be available locally and/or from a remote resource via an intervening network interface.

At optional block 202, the control circuit 101 may then capture projection data for a given patient as a function of the selected particular imaging protocol.

At block 203, the control circuit 101 accesses projection data for a given patient. The projection data may comprise, at least in part or entirely as desired, cone-beam computed tomography images. Those skilled in the art will understand that cone-beam computed tomography uses a cone-shaped X-ray beam to create a three-dimensional image of a patient's body part(s) (such as a tumor or organ-at-risk). In a typical application setting, a scanner rotates around the patient and captures a series of X-ray images from different angles. These images are processed to yield the three-dimensional image(s). The accessed projection data may have been captured as described above or may have been made available in some other way of choice.

At block 204, the control circuit 101 processes the aforementioned accessed projection data and generates a plurality of corresponding different images. In this illustrative example, the control circuit 101 does this processing, in whole or in part, as a background task. A background process will be understood to comprise a computer process that runs behind the scenes (i.e., in the background) and typically without user intervention or even a direct user interface. As one example in these regards, it will be presumed for the sake of this example that the control circuit 101 processes the projection data in the background while executing in the foreground a radiation therapy workflow process.

By one approach, the control circuit 101 generates the plurality of different images by, at least in part, selecting a plurality of reconstruction methods from amongst a plurality of available reconstruction methods. Reconstruction refers to the process of generating a two-dimensional or a three-dimensional image of an object from a set of X-ray projection images that are taken from different angles. There are various known methods for reconstructing X-ray projection data, including but not limited to, filtered back-projection and various iterative reconstruction algorithms. The present teachings are not overly sensitive to any particular selection in these regards. That said, by one approach, a particular reconstruction method may be selected as a function of one or more foreground processes being executed by the control circuit 101. For example, a particular reconstruction method may be selected that is especially compatible or appropriate with one or more steps of the foreground radiation therapy workflow process.

These teachings will accommodate automatically generating any of a wide variety of image types including, but not limited to, at least one, three, five, or any selected number of the following image types from the projection data:
an image presenting directly reconstructed relative electron density for photon treatment dose calculation and for kV imaging dose calculation;
an image presenting directly reconstructed stopping power ratio for proton or electron treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use; and
an image highlighting at least one patient implant.

It will therefore be understood that these images can be different from one another in ways beyond merely corresponding to different fields of view of a given target. Some images, for example, can directly represent a physical property such as relative electron density, proton stopping power, and so forth while other images might provide an interpretation of a physical property (as typifies, for example, a virtual non-contrast image).

At block 205, the control circuit 101 stores the aforementioned plurality of different images to provide corresponding stored images. For example, the control circuit 101 may store some or all of these images in the aforementioned memory 102. This storage activity may also occur as a background process. By one approach the images are each stored in seriatim fashion as they are rendered in a stack that is accessible as described below. By another approach, these images are stored locally until all of the images are complete, at which point they can be transferred in bulk to an accessible stack.

At block 206, the control circuit 101 receives a request that corresponds to at least one item of task-supportive content that corresponds to a particular radiation therapy workflow step. This request may be sourced, for example, by a radiation therapy workflow process that is operating in the foreground. By one approach, the aforementioned request may present a request for a specific type of image that is appropriate or even necessary for the completion of the corresponding particular radiation therapy workflow step. In lieu of the foregoing, or in combination therewith, the request may more generally characterize the workflow step itself such that the request may not itself specify a particular image type but which instead leaves that selection to the control circuit 101.

At block 207, and in response to the aforementioned request, the control circuit 101 accesses the aforementioned stored images to select at least one particular image that corresponds to the particular radiation therapy workflow step; i.e., an image that will suit the requirements of that particular step.

At optional block 208, if desired, these teachings will accommodate post-processing the selected image to generate at least one post-processed particular image. Such post-processing (coupled, by one approach, with a particular reconstruction method) can serve, for example, to yield an image that facilitates/enables a particular task at hand to be optimally performed. The aforementioned request may include information that specifies or at least aids in the selection of a particular post-processing activity.

At block 209, the control circuit 101 transmits the at least one particular image that corresponds to the particular radiation therapy workflow step (and/or the aforementioned post-processed image when available) to the foreground activity such that the latter can make use of the transmitted information to carry out radiation therapy workflow step.

So configured, the image provided can comprise a consistent representation of the task-supportive content regardless of what image-capture modality was employed to capture the projection data that was used to generate the at least one particular image. (As used herein, the word consistent means that the foreground process receives a same type of image (such as, for example, an electron density image) for a particular task at hand regardless of the original data-capture modality (for example, regardless of whether computed tomography or cone beam computed tomography was utilized).

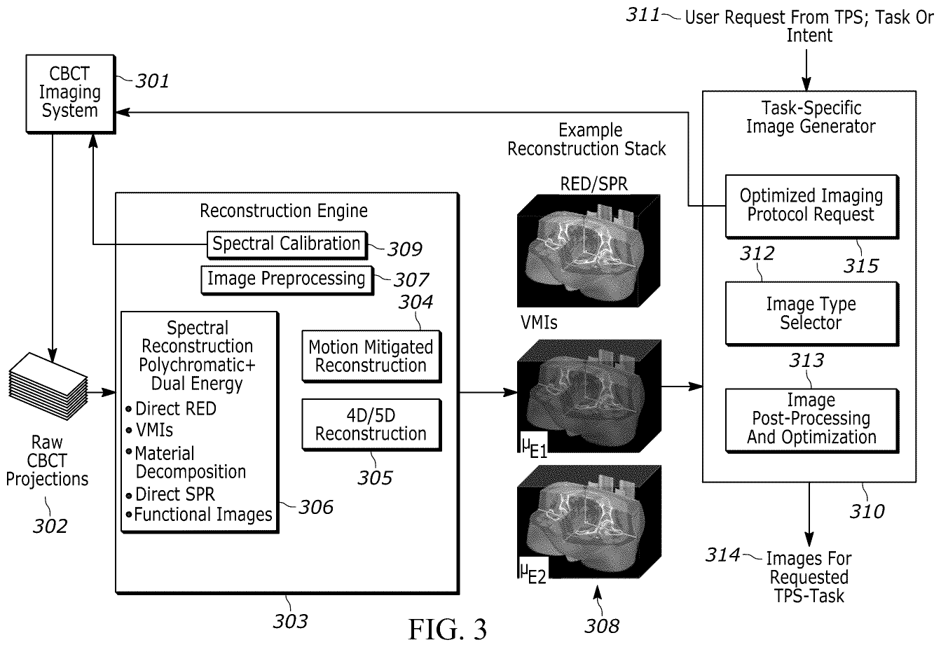

Referring now to FIG. 3, a more specific example that accords with these teachings will be provided. It shall be understood that the details in this example are intended to serve an illustrative purpose and are not intended to suggest any particular limitations with respect to these teachings.

In this example, a cone-beam computed tomography imaging system 301 captures raw cone-beam computed tomography projections 302. A reconstruction engine 303 receives those projections 302 to ultimately output a stack of reconstruction images 308. This stack 308 represents a general view that is readily available upon request. The reconstruction engine 303 itself can comprise, for example, motion-mitigated reconstruction 304, 4D/5D reconstruction 305, and/or spectral reconstruction 306. In this example the reconstruction engine 303 will also accommodate spectral calibration 309 (for use with the cone-beam computed tomography imaging system 301) and image preprocessing 307 as desired.

FIG. 3 also presents a task-specific image generator 310. This task-specific image generator 310 receives user requests 311 corresponding to a radiation therapy workflow step for one or more step-supportive images. The task-specific image generator 310 includes an image type selector 312 that selects a particular image from the aforementioned reconstruction stack 308. In particular, the selected image is particularly suited to support the needs of the workflow step corresponding to the aforementioned request 311.

In this example the task-specific image generator 310 also includes an image post-processing and optimization capability 313 to further adjust a selected image in order to even better suit that image for the intended use. These teachings will accommodate a variety of approaches in these regards. For example, such post-processing may comprise applying a (deep learning) algorithm to extend the field of view in axial but also in cranial-caudal regions to allow for dose calculation in adaptive therapy (where the extended part, which was not acquired in the cone-beam computed tomography session, is used for sufficient backscatter material in photon dose calculation), applying a (deep learning) algorithm to further improve image quality and relative electron density consistency for dose calculation on cone-beam computed tomography images, or even applying an algorithm to further mitigate motion artifacts in the reconstructed image (in contrast to a motion mitigated image reconstruction algorithm). Post processing may also serve to clean up at least some artifacts, such as unexpected air bubbles that may impact the dose calculation performance of the acquired cone-beam computed tomography image for an adaptive session. As yet another example, a potential post-processing step could comprise deriving the deformation vector field of today's cone-beam computed tomography information to be used for image comparison purposes of already acquired cone-beam computed tomography/computed tomography images to track anatomy and indicate potential re-planning and to track dosing that is based on the fractions applied in the past.

The selected (and possibly post-processed) image is then output 314 and provided as a response to the aforementioned request 311.

Accordingly, it can be seen that, for a specific requested task, the task-specific image generator 310 decides which of the images from the stack 308 to choose and what type of post-processing 313 to perform. For example, contouring might require different energy levels for corresponding virtual monoenergetic images; an optimal image might be a virtual monochromatic image at an energy that assures the best performance of, for example, an auto-contouring score or that best reflects a previously learned system (or user) preference.

Also illustrated in FIG. 3 is an optimized imaging protocol request capability 315 of the task-specific image generator 310 such that particular instructions in such regards can be provided to the cone-beam computed tomography imaging system 301.

So configured, these teachings can help to facilitate an automatic image data processing workflow that reflects a user's intentions and that provides high-quality image data tailored to support each of a variety of particular tasks in the planning process. Such a workflow can also help to reduce or avoid burdensome and error-prone user interactions within the treatment planning system (such as a selection of calibration curves to convert computed tomography numbers to relative electron density for dose calculations).

Notwithstanding that a combination of both computed tomography and cone-beam computed tomography techniques are often used as imaging modalities in adaptive radiation treatment planning, these teachings can also help to ensure that task-specific images are generated in a consistent way regardless of the initial modality. As a result, the user can expect high-quality image representation from both computed tomography and cone-beam computed tomography images.

Those skilled in the art will recognize that a wide variety of modifications, alterations, and combinations can be made with respect to the above-described embodiments without departing from the scope of the invention. As one example in these regards, these teachings are applicable in applications other than radiation treatment planning (such as tasks related to adaptive radiation therapy use cases such as tracking and treatment response analysis). As another example, these teachings will accommodate providing for real-time image processing at a time of need for at least some images (especially when that processing is not particularly computationally intensive) rather than pre-processing all possible images. Accordingly, such modifications, alterations, and combinations are to be viewed as being within the ambit of the inventive concept.

What is claimed is:

1. An apparatus, comprising:
a control circuit configured to:
access projection data for a given patient;
background process the projection data to generate a plurality of different images;
store the plurality of different images to provide stored images;
receive a request that corresponds to at least one item of task-supportive content that corresponds to a particular radiation therapy workflow step;
access the stored images to select at least one particular image that corresponds to the particular radiation therapy workflow step;
transmit the at least one particular image that corresponds to the particular radiation therapy workflow step, wherein the at least one particular image comprises a consistent representation of the task-supportive content regardless of what image-capture modality was employed to capture the projection data used to generate the at least one particular image.

2. The apparatus of claim 1 wherein the projection data comprises, at least in part, cone beam computed tomography images.

3. The apparatus of claim 2 wherein the projection data consists entirely of cone beam computed tomography images.

4. The apparatus of claim 1 wherein the control circuit is configured to background process the projection data during a radiation therapy workflow process.

5. The apparatus of claim 1 wherein the plurality of different images include at least one of:
an image presenting directly reconstructed relative electron density for photon treatment dose calculation and for kV imaging dose calculation;
an image presenting directly reconstructed stopping power ratio for proton or electron treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use;
an image highlighting at least one patient implant.

6. The apparatus of claim 1 wherein the plurality of different images includes at least three of:
an image presenting directly reconstructed relative electron density for photon or electron treatment dose calculation and for kV imaging dose calculation;
an image presenting directly reconstructed stopping power ratio for proton treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use;
an image highlighting at least one patient implant.

7. The apparatus of claim 1 wherein the plurality of different images includes at least five of:
an image presenting directly reconstructed relative electron density for photon or electron treatment dose calculation and for kV imaging dose calculation;
an image presenting directly reconstructed stopping power ratio for proton treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use;
an image highlighting at least one patient implant.

8. The apparatus of claim 1, wherein the control circuit is further configured to:
select a particular imaging protocol for a cone beam computed tomography imaging system from amongst a plurality of available imaging protocols, such that the projection data is captured as a function of the particular imaging protocol.

9. The apparatus of claim 1, wherein the control circuit is configured to background process the projection data to generate the plurality of different images by, at least in part, selecting a plurality of reconstruction methods from amongst a plurality of available reconstruction methods.

10. The apparatus of claim 1, wherein the control circuit is further configured to:
post-process the at least one particular image that corresponds to the particular radiation therapy workflow step to generate at least one post-processed particular image; and
wherein the control circuit is configured to transmit the at least one particular image that corresponds to the particular radiation therapy workflow step by transmitting the at least one post-processed particular image that corresponds to the particular radiation therapy workflow step.

11. A method, comprising:
by a control circuit:
accessing projection data for a given patient;
background processing the projection data to generate a plurality of different images;
storing the plurality of different images to provide stored images;
receiving a request that corresponds to at least one item of task-supportive content that corresponds to a particular radiation therapy workflow step;
accessing the stored images to select at least one particular image that corresponds to the particular radiation therapy workflow step;
transmitting the at least one particular image that corresponds to the particular radiation therapy workflow step, wherein the at least one particular image comprises a consistent representation of the task-supportive content regardless of what image-capture modality was employed to capture the projection data used to generate the at least one particular image.

12. The method of claim 11 wherein the projection data comprises, at least in part, cone beam computed tomography images.

13. The method of claim 12 wherein the projection data consists entirely of cone beam computed tomography images.

14. The method of claim 11 wherein background processing the projection data comprises background processing the projection data during a radiation therapy workflow process.

15. The method of claim 11 wherein the plurality of different images include at least one of:
an image presenting directly reconstructed relative electron density for photon treatment dose calculation and for kV imaging dose calculation;
an image presenting directly reconstructed stopping power ratio for proton or electron treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use;
an image highlighting at least one patient implant.

16. The method of claim 11 wherein the plurality of different images includes at least three of:
an image presenting directly reconstructed relative electron density for photon treatment dose calculation and for kV imaging dose calculation;
an image presenting directly reconstructed stopping power ratio for proton or electron treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use;
an image highlighting at least one patient implant.

17. The method of claim 11 wherein the plurality of different images includes at least five of:
an image presenting directly reconstructed relative electron density for photon or electron treatment dose calculation and for kV imaging dose calculation;
an image presenting directly reconstructed stopping power ratio for proton treatment dose calculation;
a post-processed virtual monoenergetic image, with energy selected for optimal visibility of structures to be contoured;
an image for assessing functional organs at risk dosimetry;
a virtual non-contrast image;
an effective atomic number image;
a motion mitigated image;
a phase binned 4D-cone beam computed tomography image tailored to a particular intended use;
an amplitude binned 4D-cone beam computed tomography image tailored to a particular intended use;
a 5D-cone beam computed tomography image tailored to a particular intended use;
an image highlighting at least one patient implant.

18. The method of claim 11, further comprising:
selecting a particular imaging protocol for a cone beam computed tomography imaging system from amongst a plurality of available imaging protocols, such that the projection data is captured as a function of the particular imaging protocol.

19. The method of claim 11, wherein background processing the projection data to generate the plurality of different images comprises, at least in part, selecting a plurality of reconstruction methods from amongst a plurality of available reconstruction methods.

20. The method of claim 11, further comprising:
post-processing the at least one particular image that corresponds to the particular radiation therapy workflow step to generate at least one post-processed particular image; and
wherein transmitting the at least one particular image that corresponds to the particular radiation therapy workflow step comprises transmitting the at least one post-processed particular image that corresponds to the particular radiation therapy workflow step.
